(19) United States
(12) Patent Application Publication
(10) Pub. No.: US 2006/0258935 A1
(43) Pub. Date: Nov. 16, 2006

(54) System for autonomous robotic navigation

(76) Inventors: John Pile-Spellman, New York, NY (US); Sundeep Mangla, New York, NY (US)

Correspondence Address: KENYON & KENYON LLP, ONE BROADWAY, NEW YORK, NY, 10004, US

(21) Appl. No.: 11/128,122

(22) Filed: May 12, 2005

(51) Int. Cl. A61B 5/05

(52) U.S. Cl. 600/416

(57) ABSTRACT

The present invention provides systems and corresponding methods for autonomous robotic navigation that are capable of obtaining a volumetric image data set of the structure having a lumen, defining a volume of interest between a first point within the structure having a lumen and a second point within the structure having a lumen, creating a virtual navigation pathway for navigating a navigable device with a robotic device between the first and second points, registering the navigable device within the volume of interest, and navigating the navigable device iteratively along the virtual navigation path with the robotic device.

### BACKGROUND OF THE INVENTION

The present invention generally relates to robotic systems. In particular, the present invention relates to robotic systems for navigating autonomously in a three-dimensional space.

Recent advances in imaging, computer reconstruction, robotics, and nanotechnologies have been incorporated into medical and surgical applications, including brain biopsies, orthopedic joint surgery, closed-chest coronary artery bypass grafting, ophthalmologic microsurgery, and surgical training and simulation. Examples of such advances can be seen in U.S. Pat. Nos. 5,817,022, 5,868,673, 6,246,898, 6,298,259, 6,380,958, 6,459,924, and 6,490,475, and United States Published Patent Application 2001/0025183, each of which are hereby incorporated herein by reference in their entirety.

There have also been dramatic improvements in the field of endovascular therapies, especially with respect to endovascular techniques and tools, for the adjunctive and definitive management of many vascular and non-vascular pathologic diseases, such as atrial fibrillation, septal defects, aneurysms, arteriovenous malformations, etc. As endovascular therapies continue to evolve, the techniques and devices become increasingly more complex, and demand from the surgeon a great degree of dexterity and accuracy with respect to maneuvering the endovascular devices, which typically may only be obtained after extensive and prolonged training and clinical experience. In certain therapies, the required degree of dexterity and accuracy may even be greater than that possessed by most experienced surgeons. There is therefore a need for systems that accurately navigate or maneuver endovascular devices with the accuracy necessary to carry out these advanced endovascular therapies.

### SUMMARY OF THE INVENTION

The present invention provides systems and methods for autonomous robotic navigation of a navigable device through a structure having a lumen, hereinafter referred to as a luminal structure. The term “navigable device” is used herein to generally include any device that may be manipulated or navigated within a structure having a lumen, such as endovascular or extravascular devices, including but not limited to catheters, probes, cameras, endoscopes, etc. Various luminal structures are amenable to the robotic navigation described herein, including rooms, confined spaces, pipes, corridors, anatomic structures, organs, etc. In one aspect, the present invention provides a computer system for use in autonomous robotic navigation that includes a computing device connected to an imaging device capable of producing a volumetric image data set of a luminal structure. The computing device includes software that when executed obtains the volumetric image data set of the luminal structure, and creates a virtual navigation pathway for use in navigating a navigable device with a robotic device through at least a portion of the luminal structure. In one embodiment, a volume of interest is defined between a first point and a second point within the luminal structure in which instance the virtual navigation pathway is created between the first and second points, which may be, for example, an entry point for the navigable device to enter into the luminal structure and a target point, respectively.

The virtual navigation pathway may be a path that represents the shortest distance between the first and second points or a path that is a predetermined distance from the boundaries or walls of the volume of interest. The virtual navigation pathway may also account for variables that may affect navigation within the luminal structure, such as stenotic areas, flow, pressure, and viscosity of fluid present within the luminal structure, distensibility of the walls of the luminal structure, and any limits imposed by the navigable device. In one embodiment, the virtual navigation pathway is defined as a set of meridian centerlines of intraluminal segments that make up the volume of interest.

In another embodiment, the navigable device is registered within the volume of interest and is iteratively navigated with the robotic device along the virtual navigation path. Iterative navigation, according to one embodiment, entails moving the navigable device incrementally along the virtual navigation pathway, re-imaging the luminal structure or a portion thereof, reconstructing the volume of interest and the virtual navigation pathway, and re-registering the navigable device within the reconstructed volume of interest. The iteratively navigated steps are generally repeated at least periodically or on a continuous basis to produce a real time volumetric representation of luminal structure and the navigable device during navigation. A comparison between an actual navigation path of the navigable device and the virtual navigation path may also be performed and movement of the navigable device may then account for any deviation between the actual and virtual navigation paths.

Additional aspects of the present invention will be apparent in view of the description that follows.

### BRIEF DESCRIPTION OF THE FIGURES

FIG. 1 is a block diagram of a system for autonomous robotic navigation according to one embodiment of the invention.

FIG. 2 is a flow chart of a method for autonomous robotic navigation according to one embodiment of the invention.

FIG. 3A-3B depict a structure having a lumen represented as a three dimensional data.

### DETAILED DESCRIPTION OF THE INVENTION

The present invention provides a system for autonomously navigating or manipulating a navigable device, such as an endovascular device, including a catheter or probe, through a luminal structure, such a subject's anatomical structure. Moreover, the term “subject” is used to denote any organism having luminal anatomical structure, human or otherwise. Although the present invention may be described by way of example in relation to endovascular procedures, it is understood that the present invention is applicable to other types of procedures and in other, non-medical fields, and is therefore not limited thereto.

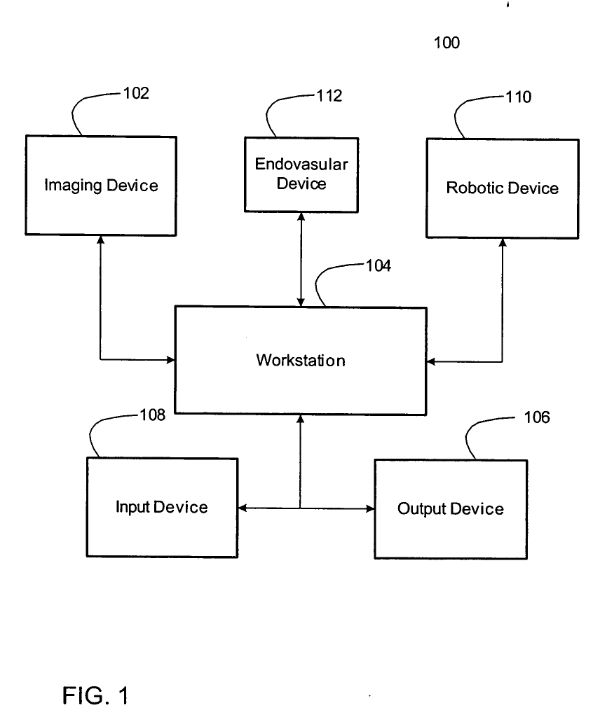

Referring to FIG. 1, a system 100 for autonomously navigating a navigable device through a luminal structure includes an imaging device 102 communicatively connected to a workstation 104. The imaging device 102 is any device that may be used to obtain or produce an image or image data of a luminal structure for use in defining the volume of interest or the boundaries of the luminal structure. The imaging device 102 may therefore be a magnetic resonance imaging device, a helical spiral computed tomographic imaging device, or any other means for producing a three-dimensional volumetric image or image data set of a luminal structure.

A workstation 104 is generally any type of computing device, such as a Vitrea 2, with software associated therewith, e.g., accessible locally from a computer readable medium or remotely in a client/server environment, that when executed provides the functional aspects of the invention as described herein, such as to define a volume of interest, compute a virtual navigation, provide a control signal for navigating a navigable device, such as an endovascular device 112, thorough a luminal structure, etc. The workstation 104 is also adopted to receive volumetric image data from the imaging device 102 to define and reproduce a volumetric image of the luminal structure. The workstation 102 may further be connected to an input device 108, such as an alphanumeric keyboard, mouse, light pen, etc. and an output device 106, such as a CRT or LCD monitor, etc., which enable a user to interface with the system 100.

In one embodiment, system 100 includes a robotic device 110 communicatively connected to the workstation 104. In this instance, the workstation 102 provides the control signal to drive the robotic device 110. The control signal generally provides the instruction or instructions that is used by the robotic device 110, for example, to navigate a navigable device, such as an endovascular device 112, through a luminal structure or to manipulate the navigable device within the structure, or a combination thereof. For instance, the control signal may provide instruction for the navigation of a catheter or other probe through a subjects' vasculature and to expand a balloon catheter at a stenosis.

The control signal may be provided to various types of robotic devices 110, including robotic devices capable of movement between two to six degrees of freedom. In one embodiment, the robotic device 110 is capable of producing two degrees of freedom, such as rotational and a longitudinal or translational degree of freedom. This embodiment is particularly suited for coaxial endovascular interventions, e.g., to navigate an endovascular device 112 through a subject's vasculature. Movement in the rotational degree of freedom generally provides torque to rotate the endovascular device 112, whereas movement in the longitudinal degree of freedom provides force to move the endovascular device 112 coaxially with respect to the blood vessels either forward into the luminal structure toward a target point or outward to withdraw the endovascular device 112 from the luminal structure away from the target point. The control signal may further provide instruction to execute certain endovascular techniques at the target site, such as to expand a balloon catheter.

In one embodiment, the robotic device 110 includes at least two motors capable of being independently operated to produce 360 degrees of rotational movement and at least 300 mm of translational movement. A catheter may be registered with the two motors to provide independent or simultaneous manipulation of the catheter via the two motors in the two degrees of freedom. The robotic device 110 may therefore manipulate standard commercially available or custom navigable devices, such as endovascular devices 112.

In one embodiment, the robotic device 110 manipulates a device that includes therein sensors that provide a signal to either the robotic device 110 or the workstation. For example, the device may be an endovascular device 112 that includes at least one sensor, such as a pressure sensor or sensors, therein. The pressure sensor generally provides a signal that indicates when the endovascular device 112 has come into contact with a subject's vasculature and the force exerted on the vasculature. The system may then, based on the signal, provide the appropriate control signal to generate an appropriate response thereby preventing injury to or perforation of the vascular tissue. The navigable device being manipulated, e.g., with the robotic device 110, is preferably radio opaque or contains radio opaque markers thereon that allow the imaging device 102 to image and co-register the navigable device within the luminal structure. The location of the navigable device will generally be used to determine whether or not the device is being navigated along a computed virtual navigation path and to take corrective action as necessary to follow the desired navigation path.

The system 100 may also be adopted to obtain or determine data with regard to the luminal structure that is relevant to navigating the navigable device within the luminal structure. For example, where the system is adopted for endovascular navigation, the relevant data may include blood flow, pressure, viscosity, distensibility of vessels, etc., thereby allowing the system to manipulate the endovascular device 112 accounting for such data. For instance, distensibility will allow the system to determine the maximum pressure that particular vascular tissue will accept without injury and manipulate the endovascular device 112 accordingly based on the distensibility data. Blood flow and pressure may, for example, be used to determine if the device is restricting flow through the vascular tissue and to take corrective action as necessary. The data may be obtained in real time based on actual measurements, statistical interpretations of actual measurements, or a combination thereof.

The system 100 may also be includes means for a user to intervene in the navigation of the navigable device. Intervention, for example, may be accomplished with an override button or switch accessible at the workstation 104, near the robotic device 110, or a combination thereof. Alternatively or in combination, the system 100 may include means for a user to control the robotic device 110 remotely, such as with a joystick, control pad, mouse, etc. In one embodiment, the system 100 provides an alarm, such as an audible or visual alarm, which may be used to signal a user for possible user intervention. An alarm may be triggered in a variety of circumstances. For example, an alarm may signal deviation between the actual and virtual navigation paths that is greater than a threshold or maximum allowed deviation. The threshold or maximum deviation generally depends on the size or diameter of the luminal structure through which the navigable devices is being navigated or manipulated. A larger deviation may be tolerated, for instance, for a luminal structure having a large diameter, such as through the aorta, in contrast to a structure having a smaller diameter, such as the femoral artery. The alarm may similarly be triggered based on data obtained by the system 100, for example, during navigation, such as data regarding pressure on the navigable device, fluid flow, fluid pressure, and fluid viscosity, distention or perforation of the walls of the structure, etc., which when interpreted may indicate a need for user intervention.

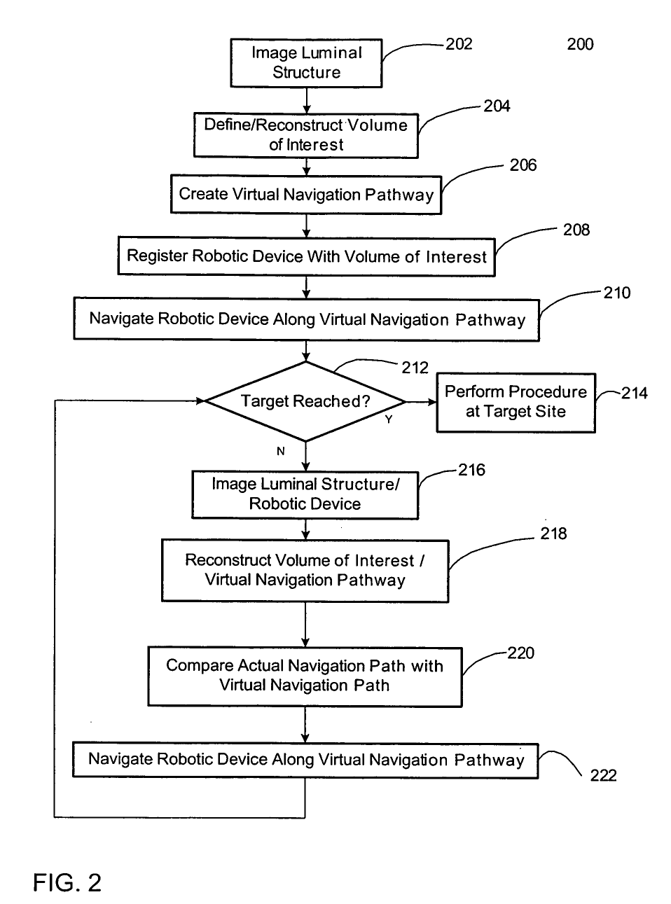

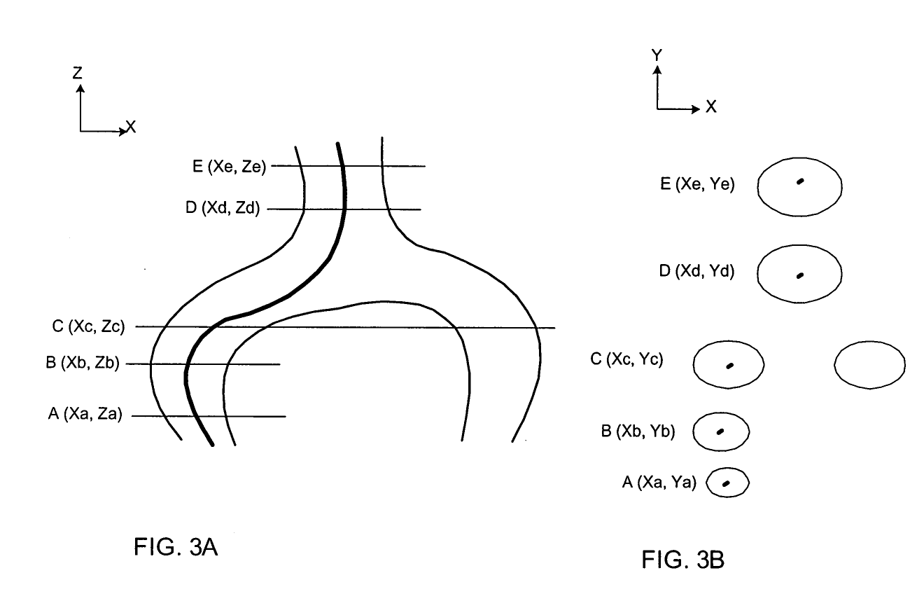

Referring to FIG. 2, an autonomous robotic navigation method 200 generally begins by imaging a luminal structure, step 202, such as a subject's vasculature. Imaging is used to generally produce a volumetric image or volumetric image data set of the luminal structure. The volumetric image/data generally defines the boundaries of the luminal structure, as shown in FIG. 3A and FIG. 3B, in a three dimensional (x, y, z) coordinate system. Thus, in accordance with at least one embodiment, points on the luminal structure are defined by its x, y, and z coordinates and the volumetric image is defined by a set of points on the luminal structure. The volumetric image/data may define the boundaries of the luminal structure in other coordinate systems, such as in a polar coordinate system. The origin of the coordinate system may be any suitable reference, including a point on the luminal structure.

The image or image data set of the luminal structure may be produced at any time prior to the commencement of the robotic procedure. Imaging is generally timely based on the mobility and flexibility of the luminal structure. For example, imaging a relatively immobile/inflexible network of pipes may occur at any time prior to robotic navigation, whereas imaging vascular structure for endovascular interventions should occur closer to the planned intervention.

Once the volumetric image data set is produced, e.g., with the imaging device 102, the image data set is transferred to the workstation 104 where a volume of interest may be defined and a three-dimensional representation of the volume of interest reconstructed, step 204. The volume of interest is generally the functional segment of the luminal structure between a point of entry and a target point. For example, where the system is adopted for autonomous robotic navigation of an angioplasty/balloon catheter to a stenosis in a cardiac artery, the volume of interest will generally include the point of entry, typically the femoral artery, to a point on the target artery beyond the stenosis. This step may be performed manually, such as by a surgeon specifying the two points via an interface provided on the workstation that displays a three dimensional image reconstruction of the luminal structure, automatically based on computer interpretation of the subject's vascular anatomy, or a combination thereof wherein the computer suggests a point of entry and locates a target location by identifying a potential stenosis and the user may either accept or override these suggestions.

A virtual navigation pathway within the volume of interest may then be created or defined, step 206. The virtual navigation pathway is an ideal or preferred navigation path. The virtual navigation pathway may, for example, be the shortest path between the entry and target point or a path that essentially follows a line that is at a specified or predetermined distance from the walls of the volume of interest. In one embodiment, the ideal navigation pathway is created while accounting for stenotic vessels that may restrict navigation of the navigable device there through, or other variables that may affect navigation within the luminal structure, such as fluid flow, pressure, viscosity, distensibility, limits of the navigable device (flexibility), etc. The variables that may affect navigation may be obtained in near real time directly from the luminal structure, e.g., at about the same time as the imaging of the luminal structure, or may be extrapolated based on obtained and known data, or a combination thereof. This step may also be performed manually, such as by a surgeon specifying the navigation points in the three dimensional image reconstruction of the luminal structure, automatically based on computer interpretation of the vascular anatomy, or a combination thereof wherein the computer suggests navigation points at various points along intraluminal segments of the volume of interest that the user may either accept or override by providing alternate navigation points or instruction.

The navigation pathway may be defined as a line or set of lines connected at their endpoints, equal or varying in lengths, which follow the ideal path or preferred path. In one embodiment, the navigation pathway is defined as a set of median centerlines of the intraluminal segments of the volume of interest to form a skeleton. The skeleton may be formed based on morphological thinning algorithms. The skeleton or set of intraluminal line segments may be smoothed using a three-term moving average filter to compensate for artifacts crated by relatively low pixel resolution along the z-axis. Although the present invention may be described in relation to this particular method of defining a navigation pathway, it is understood that various other techniques may be used to define the pathway, and is thus not limited to any one particular technique.

The robotic device 110, and, if applicable, the navigable device, are registered within the volumetric image data set, particularly with respect to the volume of interest, step 208. In one embodiment, where the system 100 is adopted for endovascular use, registration of the endovascular device 112 is accomplished by introducing the endovascular device 112 in the relevant anatomy, such as the femoral artery, imaging the device 112 to produce an image data set of the endovascular device 112, and identifying, either automatically or manually, the endovascular device within the volumetric image data set of the volume of interest. The initial endovascular device data set will serve as the starting point for device navigation. In one embodiment, all the volumetric data sets of the luminal structure and the navigable device, including data sets produced subsequent to the initial data sets are obtained in real time.

The navigable device may then be iteratively navigated or moved with the robotic device 110 incrementally along the virtual navigation path, step 210, until the target is reached, step 212, and, if applicable, the navigable device manipulated within the luminal structure. The increments of navigation may be a fixed distance, for example, 1-100 mm, in variable increments corresponding to the set of intraluminal line segments or navigation points, or increments based on based on a real time comparison between the actual navigation path and the virtual navigation path.

Iterative navigation will generally be accomplished by re-imaging, step 216, and reconstructing the volume of interest, the virtual navigation pathway, and the robotic device 110 or navigable device in the volumetric model of the luminal structure to account for movement of the volume of interest, which may cause a shift in the virtual navigation pathway, and the robotic device 110 or navigable device. These steps will preferably be repeated, continuously or otherwise, and in real time to provide, for example, a reconstructed representation of an endovascular intervention that is essentially a true representation of the actual endovascular intervention as it is being performed. At least part of the autonomous aspect of the invention is provided by comparing the near actual or reconstructed path of the robotic device or navigable device with the virtual navigation path, step 220, and navigating navigable device along the virtual navigation path while accounting for any deviation between the actual and virtual navigation paths, step 222. Accordingly, autonomous navigation properly accounts for variables that may affect navigation within the luminal structure, such as fluid flow, pressure, viscosity, distensibility, etc.

In one embodiment, the system will provide a view of the reconstructed procedure, e.g., on a display device, and allow the operator to override certain maneuvers as necessary. The system may also require the operator to validate certain maneuvers, such as manipulating an endovascular device 112, e.g., expanding a balloon catheter, at the target site.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A computer system comprising a computing device communicatively connected to an imaging device capable of producing a volumetric image data set of a structure having a lumen, wherein the computing device comprises software associated therewith that when executed performs a computerized method for autonomous robotic navigation that comprises:
obtaining the volumetric image data set of at least a portion of the structure having a lumen; and
creating a virtual navigation pathway for navigating a navigable device with a robotic device through at least a portion of the structure having a lumen.

2. The system of claim 1, wherein the method comprises defining a volume of interest between a first point and a second point within the structure having a lumen and wherein the virtual navigation pathway is created between the first and second points.

3. The system of claim 2, wherein the first point is an entry point for the navigable device to enter into the structure having a lumen and the second point is a target point.

4. The system of claim 2, wherein the virtual navigation pathway represents a path that is the shortest distance between the first and second points.

5. The system of claim 2, wherein the virtual navigation pathway represents a path that is a predetermined distance from volume of interest walls.

6. The system of claim 2, wherein the virtual navigation pathway is created while accounting for variables that may affect navigation within the luminal structure.

7. The system of claim 6, wherein the variables that may affect navigation comprise at least one of stenotic areas, fluid flow, pressure, and fluid viscosity within the structure having a lumen, distensibility of the walls of the structure having a lumen, and limits of the navigable device.

8. The system of claim 2, wherein the virtual navigation pathway is defined as a set of meridian centerlines of intraluminal segments that make up the volume of interest.

9. The system of claim 2, wherein the method comprises registering the navigable device within the volume of interest and iteratively navigating the navigable device with the robotic device along the virtual navigation path.

10. The system of claim 9, wherein iteratively navigating the navigable device comprises:
moving the navigable device incrementally along the virtual navigable pathway;
reimaging the structure having a lumen;
reconstructing the volume of interest and the virtual navigation pathway; and
reregistering the navigable device within the reconstructed volume of interest.

11. The system of claim 10, wherein the steps of reimaging, reconstructing the volume of interest and the virtual navigation pathway, and registration the navigable device within the reconstructed volume of interest are accomplished in real time.

12. The system of claim 10, wherein the method comprises comparing a navigation path representing actual movement of the navigable device within the volume of interest with the virtual navigation path, and moving the navigable device along the virtual navigation path accounting for any deviation between the actual and virtual navigation paths.

13. The system of claim 1, wherein the robotic device is capable of moving the navigable device in at least two degrees of freedom.

14. The system of claim 13, wherein the at least two degrees of freedom comprise rotational and translational degrees of freedom.

15. A computer system comprising a computing device connected to an imaging device capable of producing a volumetric image data set of a structure having a lumen, wherein the computing device comprises software that when executed performs a computerized method for autonomous robotic navigation that comprises:
obtaining the volumetric image data set of the structure having a lumen;
defining a volume of interest between a first point and a second point within the structure having a lumen;
creating a virtual navigation pathway for navigating a navigable device with a robotic device between the first and second points;
registering the navigable device within the volume of interest; and
navigating the navigable device iteratively along the virtual navigation path with the robotic device.

16. A computerized method for autonomous robotic navigation comprising:
obtaining a volumetric image data set of a structure having a lumen; and
creating a virtual navigation pathway for navigating a navigable device with a robotic device through at least a portion of the structure having a lumen.

17. The method of claim 16, comprising defining a volume of interest between a point for entry of the navigable device into the structure having a lumen and a target point and wherein the virtual navigation pathway is created between the entry and target points.

18. The method of claim 17, wherein the virtual navigation pathway is defined as a set of meridian centerlines of intraluminal segments that make up the volume of interest.

19. The method of claim 17, comprising registering the navigable device within the volume of interest and iteratively navigating the navigable device with the robotic device along the virtual navigation path.

20. A computerized method for autonomous robotic navigation comprising:
obtaining a volumetric image data set of the structure having a lumen;
defining a volume of interest between a first point and a second point within the structure having a lumen;
creating a virtual navigation pathway for navigating a navigable device with a robotic device between the first and second points;
registering the navigable device within the volume of interest; and
navigating the navigable device iteratively along the virtual navigation path with the robotic device.
